(19) United States
(12) Patent Application Publication
(10) Pub. No.: US 2013/0209564 A1
(43) Pub. Date: Aug. 15, 2013

(54) Polysaccharide Particle Vaccines

(71) Applicants: Shyam M. Rele, Cary, NC (US); Andrew Murphy, Cary, NC (US); Bolyn Hubby, San Diego, CA (US)

(72) Inventors: Shyam M. Rele, Cary, NC (US); Andrew Murphy, Cary, NC (US); Bolyn Hubby, San Diego, CA (US)

(21) Appl. No.: 13/580,212

(22) PCT Filed: Feb. 22, 2011
(86) PCT No.: PCT/US11/25754

§ 371 Date: Jan. 9, 2013

Related U.S. Application Data
(60) Provisional application No. 61/306,898, filed on Feb. 22, 2010.

(51) Int. Cl. A61K 39/385

(52) U.S. Cl. CPC A61K 39/385
USPC 424/490; 424/193.1

(57) ABSTRACT

Particle compositions are prepared for use as polysaccharide particle vaccines.

### BACKGROUND

Conjugate vaccines are typically created by covalently attaching an antigen to a carrier protein. The resulting immunogen is then often applied to bacterial polysaccharides for the prevention of bacterial diseases. A drawback to using conjugate vaccines is the difficulty in chemically conjugating the antigen to the protein. Therefore, a virtual conjugate vaccine where the antigen and the protein are presented in close association to a cell of interest which does not require the direct conjugation of protein to carbohydrate would be highly desirable.

Microparticles and nanoparticles fabricated through PRINT® technology (Liquidia Technologies, North Carolina) offers microparticles and/or nanoparticles with control over their chemical composition, particle size, particle shape, surface functionality, and other physical and chemical characteristics. Use of this technology in the present invention allows for the co-packaging of antigen(s) and carrier protein(s) to stimulate an immune response. PRINT® particles have previously been described in, for example, US 2009-0028910; WO 2008/118861; WO 2009/111588, US 2009-0165320; US 2007-0275193; US 2007-0264481; US 2008-0131692; WO 2008/127455; US 2008-0181958; US 2009-0098380; and WO 2009/132206; each of which is incorporated herein by reference in its entirety. The PRINT® particles are described further herein.

### SUMMARY OF THE INVENTION

One of the objectives of the present invention is to prepare a single particle composition that can be utilized with multiple polysaccharides (PS). Alternatively, if a single particle composition does not or is not able to accommodate selected PS, then another objective is the development of a collection of particle compositions that can accommodate the PS's of interest for a given indication or target.

An aspect of the invention is an immunogenic composition comprising a molded particle comprising a substantially uniform composition, wherein the substantially uniform composition comprises an immunogenic amount of a carrier protein associated with a polysaccharide.

A particular aspect of the invention is an immunogenic composition comprising a molded particle comprising a non cross-linked composition comprising a polymer comprising between about 10-20 wt %, a protein comprising between about 60-70 wt % and a polysaccharide comprising between about 10-25 wt %.

Another aspect of the invention is a method of optimizing the performance of polysaccharide vaccines by varying a type or stoichiometry of an immunogenic polysaccharide or immunogenic protein component of a molded two dimensional array of particles and testing performance of the particles.

Another aspect of the invention is a system for fabricating a polysaccharide vaccine comprising selecting a matrix composition compatible with a protein and a polysaccharide and forming a particle from said composition by molding the composition in a polymer mold, wherein the composition comprises between about 90-99.9 wt % protein and between about 10-0.1 wt % polysaccharide.

In a particular embodiment, the polysaccharide is a pneumococcal polysaccharide.

In a particular embodiment, the carrier protein and/or the polysaccharide is in the form of a matrix.

In a particular embodiment, the particle is not cross-linked or is cross-linked.

In a particular embodiment, the immunogenic composition further comprises a matrix selected from the group consisting of a substantially non-immunogenic protein, a sugar, a polymer and a hydrophobe.

In a particular embodiment, the immunogenic composition further comprises a polymer, wherein the polymer physically constrains the carrier protein in association with the polysaccharide.

In a particular embodiment, the polysaccharide or carrier protein is adsorbed on a surface of the particle.

In a particular embodiment, the carrier protein comprises between 90 wt % and 99.9 wt % and the polysaccharide comprises between 10 wt % and 0.1 wt %.

In a particular embodiment, the carrier protein comprises ovalbumin, CRM, or HSA between 90 wt % and 99.9 wt % and the polysaccharide comprises pneumococcal polysaccharide 14 between 10 wt % and 0.1 wt %.

In a particular embodiment, the polymer comprises PLGA at about 16 wt %.

In a particular embodiment, the protein comprises ovalbumin, CRM, or HSA at about 66 wt %.

In a particular embodiment, the polysaccharide comprises pneumococcal polysaccharide 14 at about 18 wt %.

In a particular embodiment, the matrix composition comprises components and treatments that do not reduce the immunogenicity of the protein or polysaccharide.

### DETAILED DESCRIPTION

As described herein, multiple particle compositions have been fabricated and/or contemplated to form virtual conjugate PS vaccine particles. The fabricated particles generally include particles formed from a combination of a carrier protein and a PS of interest, wherein the combination is treated with a cross-linker. In an exemplary embodiment, treatment with the cross-linker occurs before the particle is harvested from the harvest array. In another exemplary embodiment, the treatment with the cross-linker occurs after the particle is harvested from the array. In another exemplary embodiment, instead of covalently cross-linking the particle, an additive is added to the particle matrix which provides a binding force (such as, but not limited to, charge-charge interaction, adsorption, hydrophobic interactions, phase separation, polymer, or the like) sufficient to retain the carrier protein in association with the PS for a desired period of time. In another exemplary embodiment, the particles of protein and PS are treated with a coating step to package the protein and PS. In yet another exemplary embodiment, the particle is formed from a non-antigen or immunogenic (also referred to herein as non-active) polysacchraride or protein substance, such as, for example a polymer, and surface adsorbed with the protein and PS of interest.

Additives may optionally be introduced into the particle matrix as necessary to maintain the protein, the PS, or the intact protein/PS particles, such as, for example, but not limited to, lipids, amino acids, hydrophobes, polymers, small molecules, fatty acids, surfactants, and the like. Other polysaccharide and/or protein mixture techniques and complexes useful in the particles of the present invention can be found in “Formation and characterization of amphiphilic conjugates of whey protein isolate (WPI)/xanthan to improve surface activity,” by A. Benichou et al., Food Hydrocolloids 21 (2007) 379-391, which is incorporated herein by reference in its entirety.

### Particle Fabrication

PRINT® Particles

In exemplary embodiments, synthetic biocompatible polymers can be included in the PRINT® particles. According to such embodiments, some examples include, but are not limited to, synthetic polypeptides containing one or more cross-linkable cysteine residues, synthetic polypeptides containing one or more disulfide groups, linear or branched chain polyalkylene glycols, polyvinyl alcohol, polyacrylates, polyhydroxyethyl methacrylate, polyacrylic acid, polyethyloxazoline, polyacrylamides, polyisopropyl acrylamides, polyvinyl pyrrolidinone, polylactide/glycolide, combinations thereof, and the like. According to further exemplary embodiments, synthetic polymers useful in combination with the particles of the invention include, but are not limited to, synthetic polyamino acids containing cysteine residues, synthetic polyamino acids containing disulfide groups, polyvinyl alcohol modified to contain free sulfhydryl groups, polyvinyl alcohol modified to contain free disulfide groups, polyhydroxyethyl methacrylate modified to contain free sulfhydryl groups, polyhydroxyethyl methacrylate modified to contain free disulfide groups, polyacrylic acid modified to contain free sulfhydryl groups, polyacrylic acid modified to contain free disulfide groups, polyethyloxazoline modified to contain free sulfhydryl groups, polyethyloxazoline modified to contain free disulfide groups, polyacrylamide modified to contain free sulfhydryl groups, polyacrylamide modified to contain free disulfide groups, polyvinyl pyrrolidinone modified to contain free sulfhydryl groups, polyvinyl pyrrolidinone modified to contain free disulfide groups, polyalkylene glycols modified to contain free sulfhydryl groups, polyalkylene glycols modified to contain free disulfide groups, combinations thereof, and the like. The biocompatible material/polymer can be biodegradable.

In an exemplary embodiment, the predetermined geometries of the immune cell-targeted micro and/or nanoparticles of the invention include substantially spherical, substantially non-spherical, substantially viral shaped, substantially bacteria shaped, substantially protein shaped, substantially cell shaped, substantially rod shaped, substantially chiral shaped, substantially a right triangle, substantially flat disc shaped or the like an exemplary embodiment, the particles have a broadest dimension less that about 100 micrometers, for example, between about 1 urn and about 50 micrometers, such as between about 50 nm and about 10 micrometers, such as between about 100 nm and about 1 micrometer, such as between about 100 nm and about 500 nm. In other embodiments, the particles can have predetermined geometric characteristics. According to some exemplary embodiments, geometric characteristics include a shape having two substantially flat and substantially parallel sides, a predetermined radius of curvature, a predetermined angle between two sides, a substantially flat surface having a predetermined width, two substantially flat surfaces, two substantially flat surfaces being substantially parallel, two substantially flat surfaces being substantially parallel and configured a controlled distance apart, two substantially flat surfaces where the two substantially flat surfaces abut with a predetermined angle, or the like, in still further exemplary embodiments, the particles are configured into spherical, sphere-like, or spherilized shapes. According to such embodiments, particles may spontaneously spherilize at or above a given temperature (relatively low melting temperatures for compositions including biological or sensitive compositions) while in the mold cavities, after harvesting from the mold cavities and while on the harvesting array, or after harvesting from the mold cavities and while in a collection or solution. In some embodiments, the shape of the particle is less critical than the control over particle chemical composition, PS ratio, carrier protein ratio or third party matrix composition ratio.

The PRINT® technology generally utilizes low surface energy molds made from materials such as silicones, perfluoro-polyether-based elastomers (PFPEs) or other hydrocarbon-based materials to replicate micro or nano sized structures on a master template. The polymers utilized in PRINT® molds are often liquids at room temperature and are often photo-chemically cross-linked into elastomeric solids that enable high resolution replication of micro- or nano-sized structures. The liquid polymer is then “solidified” while in contact with the master, thereby forming a replica image of the structures on the master. Solidification of the mold in contact with the master can take place by curing (thermally or photochemically) or by cooling down by vitrification and or by crystallization. Upon removal of the polymer mold from the master template, the polymer forms a patterned template that includes cavities or recess replicas of the micro or nano-sized features of the master template and the micro or nano-sized cavities in the cured liquid polymer can be used for high-resolution microparticle or nanoparticle fabrication. PRINT® technology enables the fabrication of monodisperse organic and inorganic nanoparticles with simultaneous control over structure (e.g., shape, size and composition) and function (e.g., surface structure).

PRINT° technology is the first general, singular method capable of forming particles that: i) are monodisperse in size and uniform shape; ii) can be molded into any shape; iii) can be comprised of essentially any matrix material; iv) can be formed under extremely mild conditions (compatible with delicate cargos); v) are amenable to post functionalization chemistry (e.g., bioconjugation of active agents and/or targeting components); and vi) which initially fabricates particles in an addressable 2-D array (which opens up combinatorial approaches since the particles can be “bar-coded”).

Technical aspects to be considered when designing a PRINT® nanoparticle carrier system include: 1) compatibility of the particle cargo or matrix materials with the polymer PRINT® mold materials, 2) particle degradation profile desired for cargo release, 3) targeting method, 4) particle modulus, and 5) the combination of points 1-4 in the formation of a prepolymer mixture that is amenable to the PRINT® process outlined herein. Cargo/prepolymer matrix compatibility can be addressed by tuning the hydrophilicity of the prepolymer matrix to match that of the cargo through judicious choice of monomers. Particle degradation and targeting are discussed herein. Modulus and robustness of the particles can be adjusted by changing the degree of cross-linking within the particle and/or the constituents of the particle that cause a physical entanglement thereof. Finally, the particle formulation can be optimized for PRINT® fabrication, if needed, by adding co-monomers or co-solvents to alter the physical properties of the monomer solution.

In an exemplary embodiment, the particles of the invention comprise a protein matrix and a PS, wherein the particles may be cross-linked. In some particular embodiments, the protein and PS particle composition are subjected to a cross-linking while in the harvest array or after collection in solution post particle fabrication. In a particular embodiment, the protein matrix is the predominant component in the particle composition. In another particular embodiment, the PS matrix is the predominant component in the particle composition. In another particular embodiment, a polymer is included in the particle composition to physically hold the polysaccharide and protein in association. In yet other particular embodiment, the polymer component is the predominant component of the particle and the protein and polysaccharide are adsorbed to the surface of the polymer particle. In another particular embodiment, the protein component is the predominant component of the particle and the polysaccharide is adsorbed to the surface of the protein particle. In another particular embodiment, the PS component is the predominant component of the particle and the protein is adsorbed to the surface of the PS particle.

In another exemplary embodiment, the particles of the present invention comprise a protein, a PS, and other third party matrix component(s), wherein the component(s) are not limited to proteins, non-immunogenic proteins, non-antigens, sugars, polymers, hydrophobic molecules, small molecules, salts, or the like. In a particular embodiment, the particles are covalently cross-linked while in another particular embodiment, the particles are not covalently cross-linked.

In another exemplary embodiment, the particles of the invention comprise a core that includes, but is not limited to, a carrier protein or a mixture of a carrier protein and third party matrix components, where the surface of the particle has been modified to reduce solubility and also contains a polysaccharide or multiple polysaccharides of interest on the surface. Surface modifications can include, but are not limited to, coating the particle surface after particle fabrication with, for example, a mixture of PS and hydrophobic molecules.

In another exemplary embodiment, the particles of the invention comprise a core that includes, but is not limited to, a carrier protein and PS or a mixture of carrier protein or proteins, PS, and third party matrix components, where the surface of the particle has been modified to reduce solubility. Surface modification can include, but is not limited to, coating the particle surface after particle fabrication and may also include components that are designed to deliver the particles to specific B-cell populations.

In another exemplary embodiment, the particles of the invention comprise a particle that contains PS and influenza hemagglutinin protein which solubility is reduced by including matrix components with sialic acid epitopes that bind to the hemagglutinin protein.

Non covalent associations of PS and carrier protein to the particle may be utilized in the present invention as an advantageous alternative to covalent binding. Exemplary embodiments include, but are not limited to, instances where the protein and the PS are associated with or bound or attached to the particle through, ionic interactions, adsorption, hydrophobic interactions, phase separation, physical entanglement, or the like.

In an exemplary embodiment, the particles comprise a PS and an agent such as, for example, a lipid or polymer, and a carrier protein adsorbed to the surface of the particle.

As used herein, third party matrices refer to additives, modifiers, emulsifiers, binders or ligands, which could be incorporated in the particle composition to impart unique and/or advantageous properties such as, for example, stability (heat/pH), homogeneity, uniform packing density, porosity, surface properties and charge. Exemplary third party matrix components include, but are not limited to, PLGA, PLA, polyanhydrides, PLGA-b-PEG's, polycaprolactone, chitosan, GRAS materials such as arabinogalactan, behenic acid, amino acids (such as L-arginine or leucine), non-immunogenic proteins (such as human serum albumin or gelatin), or poly-ionic (bio)polymers (polyacrylic acid, chitosan, heparin, hylaronic acid) where the PS and the carrier protein are premixed with such components. Such components can facilitate the encapsulation of the protein-polysaccharide.

Incorporation of low molecular-weight emulsifiers, such as those used in food products, may be used in combination with polymers such as PLGA or PLGA-like polymers. Exemplary low molecular-weight emulsifiers include, but are not limited to, soybean lecithin, glycerin fatty acid esters, sucrose, fatty acid esters and hydrophobic amino acids such as leucine.

In the case of fatty acids, the affinity of the fatty acids (both esterified and non-esterified) for proteins (such as, for example, ovalbumin, HSA, and the like) stems from the different ionic and/or hydrogen-bonding interactions that exist between the amino acid side chains and the fatty acid carboxyl carbon. These interactions can be further modulated by the size of the fatty acid chain. As an example, oleate binds more tightly than palmitate to human and murine albumins, while the reverse has been true for bovine. Additionally, the carrier protein's tertiary structure is generally stabilized by non-local interactions—most commonly the formation of a hydrophobic core (in addition to, for example, salt bridges, hydrogen bonds, disulfide bonds, and even post-translational modifications). This tertiary structure appears to serve as the basis for the basic function of the protein.

Small molecule amino acids which are hydrophobic in nature are able to fit into the interior hydrophobic binding pockets of the protein scaffold, thus imparting structural and chemical stability and properties. Leucine, isoleucine, phenylalanine and threonine are exemplary amino acids that may be used as dispersibility enhancers' in spray-dried powders for inhalation and have been known to modify the aerosolization characteristics of spray-dried particles. Additionally, there is correlation between ligand hydrophobicity and protein stabilization: the most hydrophobic ligand, isoleucine is known to cause the most significant stabilization of LIV protein.

In an exemplary embodiment, cross-linkers and charged molecules are also a part of the third party matrix.

In an exemplary embodiment, the particles are in the form of a matrix of a polysaccharide and a protein, wherein the polysaccharide:protein ratio ranges from about 1:99 to about 99:1. In alternative embodiments, the polysaccharide:protein ratio ranges from about 2:98, 3:97, 4:96, 5:95, 6:94, 7:93, 8:92, 9:91 or 10:90. In other embodiments, the polysaccharide:protein ratio ranges from about 10:90 to about 90:10, respectively, such as about 15:85, such as about 20:80, such as about 30:70, such as about 40:60, such as about 50:50, such as about 60:40, such as about 70:30, or such as about 80:20.

In an exemplary embodiment, high molecular weight solutions of PVP/PEG are used for harvesting and cross-linking to keep protein particles insoluble for aqueous processing steps. In other exemplary embodiments, dissolution inhibition techniques include the use of cross linkers (such as, for example, but not limited to, glutaraldehyde, glutamic acid, homo-bifunctional NHS and imidates), hydrophobic molecules (such as, but not limited to, leucine, lipids and fatty acids), selected particle coating polymers (such as, but not limited to, GRAS, PLGA, others), lipids, and non-covalent interactions (such as, but not limited to, charge, albumin and hydrophobe).

Suitable polysaccharides for use in the invention include bacterial polysaccharides as well as carbohydrates of cancer cells or other non-self or target cell carbohydrates. In separate particular embodiments, the polysaccharides are the pneumococcal polysaccharides (PnP) 4 and 14. In various exemplary embodiments, other PNP's and polysaccharides are selected from Pneumovax, Meningococcal, typhoid, cell surface glycolipids, glycoproteins, Haemophilus influenzae Type b (HiB), staph, chlamydia, meningococcal type B (Mening B), C. Difficile, Pseudomonas, Group A & B strep, enterotoxigenic Escherichia coli (ETEC), tuberculosis (TB), Shigella, Salmonella typhi, Botulinum, plague, Burkholderia, and the like. Exemplary polysaccharides include, but are not limited to, PnP4, PnP6B, PnP9V, PnP14, PnP18C, PnP19F, PnP23F, PnP1, PnP3, PnP5, PnP6A, PnP7F and PnP19A of Streptococcus pneumoniae.

Suitable carrier proteins for use in the invention include, but are not limited to, CRM 197, tetanus toxoid, diphtheria toxoid, hemagglutinin, meningococcal surface proteins and capsular proteins. CRM197 is an example of a non-toxic, mutant protein derived from diphtheria toxin (DT) and that has long been recognized to be a non-toxic protein and is commonly used as a carrier for conjugate vaccines. Based on its non-toxic feature, this protein has been utilized for various purposes, including as an inhibitor of heparin-binding EGF-like growth factor (HB-EGF) and as an immunological adjuvant for vaccination. Other suitable carrier proteins include, but are not limited to, Pneumococcal: PcsB—(protein required for cell wall separation of group B streptococcus) and serine/threonine protein kinase (StkP) (Intercell); S. Aureus: agglutinin-like sequence (Als), including Als3, which promotes adhesion to the endothelial cells (also for an antigen for Candida) (Novadigm), clumping Factor A (ClfA) (Wyeth/Pfizer), clumping factor B (ClfB), iron surface determinant B (IsdB), Cna-FnBP fusion protein, Poly-N-acetyl glucosamine; Meningitis B: GNA1870, also named factor H-binding protein (fHbp) or rLP-2086 (Novartis); Group B Strep: Protein Antigens c, R and X.

The variation in chemical and/or physical structure between different PS's may require different treatment in the form of particle fabrication chemistries, compositions, additives, design, or the like to maintain, optimize, or accommodate the functionalities, interaction or presentation of certain PS's. For example, as described herein, several of the seven PS's of Prevnar® include reactive groups, such as carboxylic acid groups, amine groups, phosphates/phosphorylated sugars and the like, which could be sites that cross-link or otherwise react and alter the recognition, presentation, function or the like of the PS. Moreover, in alternative embodiments, the present invention provides particle composition, size, and shape tuneability to accommodate alternative conformational epitopes of polysaccharides which may play a role in the immunogenicity and biological responsiveness of such epitopes or polysaccharides.

In an exemplary embodiment, the protein and polysaccharide are adsorbed to ‘other’ particles via techniques such as, for example, spray drying, ultrafiltration, emulsions, and the like. Compositions, components, agents, techniques and chemistries useful in the present invention are further disclosed in, for example, US 20080009606; “Theoretical stability maps for guiding preparation of emulsions stabilized by protein-polysaccharide interfacial complexes,” by Cho Y H et. al., Langmuir, 2009 Jun. 16; 25(12):6649-57; U.S. Pat. No. 7,601,381; US 20090238885; “Gel particles from spray-dried disordered polysaccharides” by Paulomi Burey et al., Carbohydrate Polymers, Volume 76, Issue 2, 17 Mar. 2009, pages 206-213; each of which is incorporated herein by reference in its entirety.

In an exemplary embodiment, a polysaccharide is adsorbed to the surface of a PRINT® particle, such as by introducing a matrix composition to the polysaccharide present in or on the harvest array which coats/encapsulates the particles. In a particular embodiment, the particles to be treated are PLGA-DC-Cholesterol particles, which results in polysaccharide-coated PLGA-DC-Chol nano/microparticles. In another embodiment, the polysaccharide can be further mixed with polymers such as, for example, PvOH, PvP, Luvitec and the like. Stated differently, a particle of a non-active composition can be harvested onto a harvest layer of or including an active composition, such as, for example, an antigen. In such an embodiment, the particle adsorbs, binds, charge interacts, chemically attaches, physically attaches, or otherwise associates with the active agent in the harvest layer and forms a vaccine.

In an exemplary embodiment, a PRINT® particle was fabricated from DC-Cholesterol/PLGA and harvested onto a thin film of polysaccharide, with or without protein or secondary adsorption of protein. The polymer diluents were harvested; there is the option of having multi-coated harvesting layers (such as, for example, a polysaccharide on top of a thin film of PVOH, hyaluronic acid, dextran, xanthan gum); and a matrix additive screen.

A third party matrix represents another exemplary embodiment, where other protein matrixes act as carriers for the polysaccharide/protein adsorbed to the particle surface. Suitable matrices include, but are not limited to: MSA/HSA/Gelatin with and/or without sugars, where MSA is mouse serum albumin and HAS is human serum albumin. Other carrier polysaccharide matrices include, for example, hylauronic acid or any binder material such as gums (such as, for example, guar gum) natural binders and derivatives such as, for example, alginates, chitosan, gelatin and gelatin derivatives. Gums and dextrans represent particular embodiments of could also be used. In an exemplary embodiment, these materials are a part of the matrix in addition to the (polysaccharide and protein) blend. In another exemplary embodiment, these materials are a part of the harvest layer. In yet another embodiment, these materials are used in combination with PLGA or PLGA-like polymers (hydrophilic-hydrophobic matrix) as a blend in certain percentages.

Polymers that could play an integral role in the particle matrix composition where covalent cross-linking is not involved include, but are not limited to, hydrophobic polymers, such as, for example, PLGA, PLA, PLLA, polycaprolactone, gelatin, agarose, agaropectin, agar, lipids, degradable PEGs, artificial proteins and polyanhydrides. The rationale for this belief is that premixing certain amounts of PLGA with the (protein and polysaccharide) blend would hold the particle together without chemically cross-linking the principle components (carrier protein and polysaccharide). PEGs are also useful as binders and matrix formers.

Hydrophilic polymers such as, but not limited to, polyvinylpyrrolidones (PVP) and plasdones are commonly used as harvest layers in the formation of molded micro and nano-particles. It is known that synthetic polymers such as Luvitec/plasdone, polyvinylpyrrolidone (PVP), acrylic acid derivatives (such as, for example, Eudragit, Carbopol) allow for outstanding film formation, initial tack and adhesion to different materials, high capacity for complex formation, good stabilizing and solubilizing capacity, insensitivity to pH changes, ready radiation-induced cross-linkability as well as good biological compatibility.

Protein denaturation is a function of the temperature and the pH of the aqueous solution. While higher temperature may generally accelerate the conjugation reaction, higher temperature also may cause heat denaturation of the proteins. While this could be true during the PRINT® particle fabrication, addition of some of these binders or additives mentioned above in elevated concentrations (such as, for example, polysaccharide or PLGA) may protect the protein from heat denaturation. Additionally, the polysaccharides (as binders)/polymers (PLGA-like) may also act as a protective reagent in preventing excessive protein denaturation and/or aggregation.

Various exemplary embodiments of the invention are directed to alternative compositions to particles cross-linked with glutaraldehyde. Particles are held together with or through ionic interactions. In a particular embodiment, the particle composition includes the following components: ovalbumin; polysaccharide; glycerol; aminoguanidine; diluent: H₂O:IPA 7:3. In another particular embodiment, the components have the following ratios: ovalbumin (10%); polysaccharide (2.5%); glycerol (10%); aminoguanidine (10%); diluent: H₂O:IPA 7:3.

In an exemplary embodiment, the particles are held together through a homo-bifunctional cross-linker combined within the matrix of the particle. In a particular embodiment, the particles have the following composition: ovalbumin; polysaccharide; glycerol; imidate linker; and diluent: H₂O:IPA 7:3. In another particular embodiment, the components have the following ratios: ovalbumin (10%); polysaccharide (2.5%); glycerol (10%); imidate linker (10%); and diluent: H₂O:IPA 7:3.

In other particular embodiments of the present invention, the particles have the following composition: PLGA—Dextran blend combined with ovalbumin; polysaccharide; glycerol; and diluent: H₂O:IPA 7:3. In another particular embodiment, the particles have the following composition in the following ratio: PLGA (1%)—Dextran (2.5%) blend combined with ovalbumin (10%); polysaccharide (2.5%); glycerol (10%); and diluent: H₂O:IPA 7:3.

In other particular embodiments of the present invention, the particles have the following composition: PLGA—Xanthan Gum blend combined with ovalbumin; polysaccharide; glycerol; diluent: H₂O:IPA 7:3. In another particular embodiment, the particles have the following composition in the following ratio: PLGA (1%)-xanthan gum (0.625%) blend combined with ovalbumin (10%); polysaccharide (2.5%); glycerol (10%); diluent: H₂O:IPA 7:3.

The immunogenic compositions of the invention can include other agents, excipients or stabilizers. For example, to increase stability or decrease non-specific uptake by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearoylphosphatidylcholine (DSPC), hydrogenated soy phosphatidylcholine (HSPC), and other related compounds. Negatively charged surfactants or emulsifiers are also suitable as additives, for example, sodium cholesteryl sulfate and the like. Similarly, the positive zeta potential of nanoparticles can be altered by adding positively charged components.

Formulations suitable for oral administration can consist of (a) liquid solutions, such as an effective amount of the particles dissolved in diluents, such as water, saline, juice, orange juice, or the like, (b) capsules, sachets or tablets, each containing a predetermined amount of the particles, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the particles in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable pharmaceutical carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Immunogenic formulations suitable for parenteral administration include aqueous and non-aqueous, isotonic sterile injection solutions, which can contain antioxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. In some embodiments, the pharmaceutical composition is formulated to have a pH range of about 4.5 to about 9.0, including for example, pH ranges of any of about 5.0 to about 8.0, about 6.5 to about 7.5, and about 6.5 to about 7.0. In some embodiments, the pH of the pharmaceutical composition is formulated to no less than about 6, including, for example, no less than about any of 6.5, 7 or about 8. The immunogenic composition can also be made to be isotonic with blood by the addition of a suitable tonicity modifier, such as glycerol.

The immunogenic compositions comprising the immune cell-targeted micro and/or nanoparticles described herein can be administered to a subject (such as human) via various routes, such as parenterally, including intravenous, intra-arterial, intraperitoneal, intranasal, intrapulmonary, oral, inhalation, intravesicular, intramuscular, intratracheal, subcutaneous, intraocular, intrathecal, or transdermal. For example, the nanoparticle composition can be administered by inhalation to target immune cells of the respiratory tract. In some embodiments, the nanoparticle composition is administrated intravenously, while in other embodiments, the nanoparticle composition is administered orally.

The immunogenic compositions of the invention provide for better biodistribution of the immune cell-targeted micro and/or nanoparticles upon administration, and additionally allow for enhanced stability. In this manner, more of the active agent is delivered at the target site.

### Data

Two in vivo studies have been conducted: an initial study using (i) OVA (non-endotox free) as the carrier protein with PnP4 and alternatively PnP14; and (ii) an adsorption based theory with a polymer particle having PnP4 and alternatively PnP14 adsorbed. In the follow-up study, the OVA (non-endotox free) protein carrier was re-tested and the study expanded to test endotox free OVA as well as HSA combined with PnP4 and alternatively PnP 14.

As shown below, the pre-boost data shows significant IgG response as well as the 1 wk post boost and 2 wk post boost.

Interestingly, boosting with soluble control PnP14 after initial prime injection of PRINT® particle shows IgG response at 1 wk and 2 wk post boost.

Post-prime titers consistently show IgG response with PRINT® particles as opposed to Prevnar® (leading prior art product). In one particle group, at two weeks post-prime a PnP14 IgG titer shows PRINT® particles having a 2-fold greater IgG response than Prevnar®. As a control injection of soluble OVA with soluble PnP14 resulted in no observable antibody titer.

In exemplary embodiments, particle compositions can include a polymer mixed with the protein/polysaccharide composition. In particular embodiments, the polymer includes a biocompatible or biodegradable polymer and can be between 0 to about 5 wt %, about 0.5 to about 5 wt %, about 0.5 to about 4 wt %, about 0.5 to about 3 wt %, about 0.5 to about 2 wt %, about 0.5 to about 1.5 wt %, about 1.5 wt % or about 1 wt %. In other particular embodiments, the protein to polysaccharide ratio can be between about 99:1, 98:2, 97:3, 96:4, 95:5, 94:6, 93:7, 92:8; 91:9, 90:10, 80:20, 70:30, 60:40, 50:50, 40:60, 30:70, 20:80, or 10:90 in the particle composition. In some exemplary embodiments, the polymer adds a physical stability to the particle and helps maintain physical association of the polysaccharide with the protein. In some embodiments, the polymer is selected as a biocompatible, biodegradable, controlled degradation, or the like polymer.

Further exemplary embodiments of the present invention include particles of a polymer combined with a protein and a polysaccharide. In a particular embodiment, the particle is not cross-linked and includes a polymer comprising 16 wt %, a protein comprising 66 wt % and a polysaccharide comprising 18 wt %. In specific embodiments of such, the polymer comprises PLGA. In specific embodiments of such, the polysaccharide comprises pneumococcus polysaccharide 14. In specific embodiments of such, the protein comprises ovalbumin. In a specific embodiment, the particle composition comprises PLGA polymer at 16 wt %, ovalbumin protein at 66 wt % and pneumococcus polysaccharide 14 at 18 wt %. In a further embodiment, the present invention includes a polysaccharide vaccine particle having a protein to polysaccharide ratio of between 3.5:1 and 3.75:1. A particular embodiment of the present invention includes a polymer-based polysaccharide vaccine particle having an ovalbumin protein to pneumococcus polysaccharide ratio of 3.67:1.

In some exemplary embodiments, the shape of the particle is less critical than control over the particle's chemical composition, PS ratio, carrier protein ratio or third party matrix composition ratio. According to some of these embodiments of polymer-based polysaccharide vaccine particles, the particles may be formed as non-discrete particles or not have or maintain strict control with respect to a three dimensional shape because, for example, the polymer is non cross-linked, not heavily cross-linked or is thermally sensitive or solvent sensitive or the like.

Further exemplary embodiments of the present invention comprise particles of a protein combined with a polysaccharide. According to the polysaccharide vaccine particles fabricated from a protein and a polysaccharide, the particles can be cross-linked during fabrication or a cross-linker can be introduced to the particles after the particles are fabricated such that a non-conjugated polysaccharide vaccine is formed. In some exemplary embodiments of such, the protein comprises ovalbumin, CRM, or HSA. In some exemplary embodiments of such, the protein comprises ovalbumin, CRM, or HSA and the polysaccharide comprises pneumococcus polysaccharide 14. In a particular embodiment of the polysaccharide vaccine particle of the present invention, the protein comprises ovalbumin in a range between about 90 and about 99.9 wt % and the polysaccharide in a range between about 10 and 0.01 wt %. After molding such a particle, the particle can be treated with a cross-linker. In a particular embodiment of the polysaccharide vaccine particle of the present invention, the protein comprises ovalbumin in a range between about 92 and about 99.9 wt % and the polysaccharide in a range between about 8 and about 0.01 wt %. After molding such a particle, the particle can be treated with a cross-linker. In a particular embodiment of the polysaccharide vaccine particle of the present invention, the protein comprises ovalbumin in a range between 93 and 99.9 wt % and the polysaccharide in a range between 7 and 0.01 wt %. After molding such a particle, the particle can be treated with a cross-linker. In a particular embodiment of the polysaccharide vaccine particle of the present invention, the protein comprises ovalbumin at 93.7 wt % and the polysaccharide comprises pneumococcus polysaccharide at about 6.3 wt %. After molding such a particle, the particle can be treated with a cross-linker. In a particular embodiment of the polysaccharide vaccine particle of the present invention, the protein comprises ovalbumin at about 96.4 wt % and the polysaccharide comprises pneumococcus polysaccharide at about 3.6 wt %. After molding such a particle, the particle can be treated with a cross-linker. In a particular embodiment of the polysaccharide vaccine particle, the protein comprises ovalbumin at about 99 wt % and the pneumococcus polysaccharide at about 1 wt %. After molding such a particle, the particle can be treated with a cross-linker. In a particular embodiment of the polysaccharide vaccine particle of the present invention, the protein comprises ovalbumin at about 99.7 wt % and the polysaccharide comprises pneumococcus polysaccharide at about 0.3 wt %. After molding such a particle, the particle can be treated with a cross-linker. In a particular embodiment of the polysaccharide vaccine particle, the protein comprises ovalbumin at about 99.97 wt % and the polysaccharide comprises pneumococcus polysaccharide at about 0.03 wt %. After molding such a particle, the particle can be treated with a cross-linker.

In an alternative particular embodiment, the protein component of the present invention comprises HSA protein at about 96.4% and the polysaccharide component comprises 3.6 wt %.

In another particular embodiment, the protein component of the polysaccharide vaccine particle comprises CRM protein at about 45 wt % and the polysaccharide component comprises pneumococcus polysaccharide 14 at about 55 wt %.

In some embodiments, the shape of the particle is less critical than the control over the particle's chemical composition, PS ratio, carrier protein ratio or third party matrix composition ratio. According to some embodiments of protein-based polysaccharide vaccine particles, because, for example, the particles are formulated with a solvent which after being removed leaves porous or soft particles or the like, the particles may be formed into non discrete particles or not have or maintain strict control with respect to a three dimensional shape.

### Examples

### Example 1

### Mouse Study 1, x-Linked Protein/Polysaccharide Particles

Solutions of each particle component were prepared by dissolving each component in water at the following concentrations: 2 mg/mL polysaccharide, 10 mg/mL CRM₁₉₇, 55 mg/mL glycerol, and 55 mg/mL ovalbumin. Solutions used to prepare particles were prepared by mixing the individual component solutions per the volumes given in the following table. These final solutions contained 3 wt % total solids. In all casting solutions, glycerol was 0.9 wt %, ovalbumin was nominally 2 wt %, and the polysaccharide (PnP4 or PnP 14) was at 0.075 wt %. Solutions containing CRM₁₉₇ were at 0.075 wt % of the toxoid.

| | | μL | | | | |
|---|---|---|---|---|---|---|
| | μL PS | CRM₁₉₇ | μL glycerol | μL OVA | μL | Total vol |
| Stock Description | (2 mg/mL) | (10 mg/mL) | (55 mg/mL) | (55 mg/mL) | water | (μL) |
| OVA + PnP14 | 112.5 | 0 | 49.1 | 110.5 | 27.9 | 300 |
| OVA + PnP14 + | 112.5 | 22.5 | 49.1 | 106.4 | 9.5 | 300 |
| CRM197 | | | | | | |
| OVA + PnP4 + | 93.8 | 18.8 | 40.9 | 88.6 | 7.9 | 250 |
| CRM197 | | | | | | |

The casting solutions were coated on 5 mil PET-raw using a #3 Mayer rod, and immediately blown with cool air to evaporate the water and create a film of protein, polysaccharide, and glycerol. The films appeared clear and uniform by eye. A Fluorocur mold with 200 nm×200 nm cylindrical cavities was filled by laminating the mold with the film and then passing through a heated nip [Temp: 205° F., Pressure: 60 psi, Speed: 2 fpm]. The PET was separated from the mold after passing through the heated nip leaving the mold cavities filled with the composition of the film. The filled mold was then laminated to a Luvitec-coated PET harvest layer by again passing through a heated nip [Temp: 205° F., Pressure: 60 psi, Speed: 2 fpm]. After cooling, the mold was peeled away from the harvest layer, resulting in the transfer of the 200 nm×200 nm particles from the cavities of the mold to the harvest layer. Particles were stored on the harvest sheet at 4° C.

Glutaraldehyde was used as a cross-linker for the molded polysaccharide/protein particles. Glutaraldehyde (70% in water) was applied to the particle array. A sheet of PET was placed on top of the harvest sheet, and a rubber roller was used to spread the glutaraldehyde across the entire array (array cross-linking). After 10 minutes, the PET sheet was removed. Particles were collected into 70 mM CNBH₃Na (aq) using a polyethylene-blade cell scraper. The particles were pelleted by centrifugation three times (30 minutes, 11500×g, 4° C.), and each time the supernatant was removed and the particles were re-suspended into 1 mL WFI. Particles were further diluted into a sterile (0.22 μm filtered) 0.1% PVOH 100k/5% mannitol vehicle prior to injection.

Adsorption particles were also tested in which either polysaccharide alone or both CRM197 and polysaccharide were adsorbed to the surface of 80 nm×320 nm cationic 95% PLGA/5% pDMAEMA base particles. Base particles were fabricated by introducing the PLGA/pDMAEMA composition into cavities in a Fluorocur mold, collecting and purifying in 0.1% PVOH 100K. The base particle was formed into six different groups of adsorption particles as follows:
1. For one group, 18.2 μg PnP4 in water was added to 728 μg base particles in suspension. The mixture was vortexed for 10 seconds and then incubated on ice for thirty minutes. Particles were diluted in a sterile 0.1% PVOH 100K/5% mannitol vehicle prior to injection. Particles were dosed to deliver 80 μg particles and 2 μg PnP4 to each animal.
2. For one group, 18.2 μg PnP4 in water was added to 1092 μg base particles in suspension. The mixture was vortexed for 10 seconds and then incubated on ice for thirty minutes. Particles were diluted in a sterile 0.1% PVOH 100K/5% mannitol vehicle prior to injection. Particles were dosed to deliver 120 μg particles and 2 μg PnP4 to each animal.
3. For one group, 18.2 μg CRM197 in water was added to 728 μg base particles in suspension. Then 18.2 μg PnP4 in water was added to this mixture. The mixture was vortexed for 10 seconds and then incubated on ice for thirty minutes. Particles were diluted in a sterile 0.1% PVOH 100K/5% mannitol vehicle prior to injection. Particles were dosed to deliver 80 ug particles, 2 μg PnP4, and 2 μg CRM197 to each animal.
4. For one group, 18.2 μg CRM197 in water was added to 1092 μg base particles in suspension. Then 18.2 μg PnP4 in water was added to this mixture. The mixture was vortexed for 10 seconds and then incubated on ice for thirty minutes. Particles were diluted in a sterile 0.1% PVOH 100K/5% mannitol vehicle prior to injection. Particles were dosed to deliver 120 ug particles, 2 μg PnP4, and 2 μg CRM197 to each animal.
5. For one group, 18.2 μg PnP14 in water was added to 1092 μg base particles in suspension. The mixture was vortexed for 10 seconds and then incubated on ice for thirty minutes. Particles were diluted in a sterile 0.1% PVOH 100K/5% mannitol vehicle prior to injection. Particles were dosed to deliver 120 μg particles and 2 μg PnP14 to each animal.
6. For one group, 18.2 mg CRM197 in water was added to 1092 mg base particles in suspension. Then 18.2 μg PnP14 in water was added to this mixture. The mixture was vortexed for 10 seconds and then incubated on ice for thirty minutes. Particles were diluted in a sterile 0.1% PVOH 100K/5% mannitol vehicle prior to injection. Particles were dosed to deliver 120 ug particles, 2 μg PnP14, and 2 μg CRM197 to each animal.

CRM197 binding to particles was tested by Bradford. All groups were determined to have greater than 84% of the CRM197 bound to the base particle. Polysaccharide binding to the base particles was tested by HPLC. All PnP4 groups were determined to have 100% of the polysaccharide bound. PnP14 groups were determined to have greater than 42% of the polysaccharide bound.

Summary of Immunogenicity from Above Particles:

Soluble polysaccharide (see Results) and naked PLGA/pDMAEMA particles with no antigen adsorbed did not generate a detectable IgG response, indicating that a T-cell dependent anti-polysaccharide antibody response and associated memory response was not achieved.

Ovalbumin+PnP14 particles generated a detectable IgG response, indicating the induction of a T-cell dependent anti-polysaccharide antibody response, resulting in antibody isotype switching and development of a memory response.

Ovalbumin+PnP14+CRM197 particles generated a detectable IgG response, indicating the induction of a T-cell dependent anti-polysaccharide antibody response, resulting in antibody isotype switching and development of a memory response.

Prevnar® generated a detectable IgG response, indicating the induction of a T-cell dependent anti-polysaccharide antibody response, resulting in antibody isotype switching and development of a memory response.

In addition, ovalbumin/polysaccharide particles were consistently capable of inducing the production of measurable titers of anti-polysaccharide IgG following a single injection (measured 4 weeks post-prime). This behavior was not observed in animals injected with Prevnar® (Pfizer Inc.) as shown in Graph I.

### Example 2

### Mouse Study 2

Casting solutions were prepared from 2 mg/mL polysaccharide in WFI, 100 mg/mL glycerol in WFI, and either lyophilized standard-grade ovalbumin, lyophilized endotoxin-free (EndoGrade) ovalbumin, lyophilized human serum albumin, and isopropyl alcohol (IPA). The two different purities of ovalbumin were used to control for the potential immunological effect of lipopolysaccharide present in standard-grade ovalbumin. Final concentrations of components were as follows:

| | Solution Components | | | |
|---|---|---|---|---|
| Protein Matrix | [IPA] | [Glycerol] | [Protein] | [PnP] |
| OVA | 12% | 4% | 6% | 0% |
| OVA (std) + PnP14 | 7.50% | 2.8% | 2.7% | 0.1% |
| OVA (E-Free) + | 3.25% | 2.50% | 1.35% | 0.05% |
| PnP14 | | | | |
| H.S.A. + PnP14 | 6% | 2.24% | 3.24% | 0.12% |
| OVA (std) + PnP4 | 6% | 2.24% | 3.24% | 0.12% |

The casting solutions were coated on 5 mil PET-raw using a #3 Mayer rod, and immediately blown with cool air to evaporate the solvent. Fluorocur mold with 200 nm×200 nm cylindrical cavities were filled by laminating the mold with the film and then passing through a heated nip [Temp: 210-220° F., Pressure: 60 psi, Speed: 2 fpm]. The PET was separated from the mold after passing through the heated nip leaving the mold cavities filled with the composition of the film. The filled mold was then laminated to a Luvitec-coated PET harvest layer by again passing through a heated nip [Temp: 210-220° F., Pressure: 60 psi, Speed: 2 fpm]. After cooling, the mold was peeled away from the harvest layer, resulting in the transfer of 200 nm×200 nm particles to the harvest layer. Particles were stored on the harvest array at 4° C.

In addition to cross-linking the particles on the harvest array, (array cross-linking), as described in Example 1, particles were also cross-linked after collection into a non-solvent for the particle matrix (solution cross-linking), as described herein. The “OVA (std)+PnP14” group as well as two of three “OVA (E-free)+PnP14” groups employed Array Cross-linking. For the remaining groups, particles were collected into IPA using a polyethylene-blade cell scraper. The particles were pelleted by centrifugation two times (20 minutes, 10000×g, 4° C.). Each time the supernatant was removed and the particles were re-suspended into IPA. The second re-suspension was to 400 μL, to which 400 μL glutaraldehyde solution (a mixture of 20 μL of 70% aq glutaraldehyde and 380 μL IPA) was added. The suspension was vortexed for 30 minutes. 800 μL 70 mM CNBH₃Na was then added and the suspension was vortexed for 30 seconds. Particles were kept at 4° C. for 10 minutes. Particles were then pelleted by centrifugation four times (20 minutes, 10000×g, 4° C.), and each time the supernatant was removed and the particles were re-suspended in 1 mL WFI. Particles were further diluted into a sterile 0.1% PVOH 100k/5% mannitol vehicle prior to injection.

### Summary of Immunogenicity for Second Animal Study

Groups injected with soluble controls consisting of PnP14, standard-grade ovalbumin co-injected with PnP14, endotoxin-free (EndoGrade) ovalbumin co-injected with PnP14, or Pneumovax-23 did not generate a detectable IgG response (see Results), indicating that a T-cell dependent anti-polysaccharide antibody response, and associated memory response, was not achieved.

Ovalbumin-only particles did not generate a detectable IgG response (see Results), indicating that a T-cell dependent anti-polysaccharide antibody response, and associated memory response, was not achieved.

Ovalbumin and HSA particle types containing PnP 14, both array- and solution-cross-linked, generated a detectable IgG response, indicating the induction of a T-cell dependent anti-polysaccharide antibody response, resulting in antibody isotype switching and development of a memory response.

Prevnar® generated a detectable IgG response, indicating the induction of a T-cell dependent anti-polysaccharide antibody response, resulting in antibody isotype switching and development of a memory response.

In addition, similar to what was observed in the first animal study, ovalbumin/polysaccharide and HSA/polysaccharide particles were consistently capable of inducing the production of measurable titers of anti-polysaccharide IgG following a single injection (measured 4 weeks post-prime). This behavior was not observed in animals injected with Prevnar® (Pfizer Inc.) as shown in Graph II (data from group 10 of the follow-up study).

Graph III shows the comparison of groups 8 and 10 from the follow-up study comparing the array cross-linking with solution cross-linking particle fabrication techniques.

### Example 3

### Cross-Linked Protein/Polysaccharide Particles for Other Vaccines

A process is carried out in substantially the same way as described in Example 1 or 2, except that the PS is selected from Pneumovax, Meningococcal, typhoid, cell surface glycolipids, glycoproteins, HiB, Staph, Chlamydia, MenB, C. Difficile, Pseudomonas, Group A & B strep, ETEC, TB, Shigella, Salmonella Typhi, Botulinum, Plague, or Burkholderia.

### Example 4

A vaccine particle can be fabricated having the following composition: PLGA 20 uL (1% w/w in DMF) is added to a combined standard grade ovalbumin (32.4 uL 5.4% w/w); polysaccharide (1.2 uL 0.2% w/w); glycerol (33.64 uL 10% w/w); diluent: H₂O:IPA 7:3. The ovalbumin:polysaccharide ratio in the final matrix composition is 96.4:3.6. The harvested array of 200×200 nm molded particles when collected in water furnished a turbid suspension of particles.

### Example 5

A vaccine particle can be fabricated having the following composition: PLGA 40 uL (1% w/w in DMF) is added to a combined standard grade ovalbumin 20 uL (10% w/w in water); polysaccharide 20 uL (2.5% w/w in water); glycerol 20 uL (10% w/w in water); 60 uL diluent: H₂O:IPA 7:3. The harvested array of 200×200 nm molded particles when collected should stimulate an immune response to the polysaccharide.

### EXPERIMENTAL AND RESULTS

Total of 13 groups involving particle compositions of the present invention and soluble controls were dosed in the follow-up study as follows.

Particles of the following groups 1, 2-12 were prepared:

| GRP | Particles | Xlink |
|---|---|---|
| 1 | Blank OVA | Solution |
| 7 | OVA (std) + PnP14 | Array |
| 8 | OVA (EU-Free) + PnP14 | Array |
| 9 | OVA (EU-Free) + PnP14 | Array |
| 10 | OVA (EU-Free) + PnP14 | Solution |
| 11 | H.S.A. + PnP14 | Solution |
| 12 | OVA (EU-Free) + PnP4 | Solution |

### Soluble Control Groups 2-6 and 13

| | Soluble |
|---|---|
| Group | controls |
| 2 | PnP14 |
| 3 | OVA (std) + PnP14 |
| 4 | OVA (EU-free) + PnP14 |
| 5 | Prevnar ® |
| 6 | PnP14 + PnP4 |
| 13 | Pneumovax (dil in veh) |

### Results

Initial Study
(Liq. 23 ELISA
(IgG))

| | | | PnP 4 | | | | | | | |
|---|---|---|---|---|---|---|---|---|---|---|
| | | | Pre-bleeds | | 4 week PP | | 1 week PB | | 2 weeks PB | |
| Group | Animal | Vaccine | GMT | Titers | GMT | Titers | GMT | Titers | GMT | Titers |
| 1 | 1 | Vehicle Control | 20 | 20 | 20 | 20 | 20 | 20 | 20 | 20 |
| | 2 | | | 20 | | 20 | | 20 | | 20 |
| | 3 | | | 20 | | 20 | | 20 | | 20 |
| 2 | 4 | 80 μg 80 × 360 nm PRINT | 20 | 20 | 20 | 20 | 20 | 20 | 20 | 20 |
| | 5 | 5% DC-Chol/PLGA, no | | 20 | | 20 | | 20 | | 20 |
| | 6 | protein | | 20 | | 20 | | 20 | | 20 |
| 3 | 7 | Soluble PnP 4, 2 μg | 20 | 20 | 20 | 20 | 20 | 20 | 20 | 20 |
| | 8 | | | 20 | | 20 | | 20 | | 20 |
| | 9 | | | 20 | | 20 | | 20 | | 20 |
| | 10 | | | 20 | | 20 | | 20 | | 20 |
| | 11 | | | 20 | | 20 | | 20 | | 20 |
| | 12 | | | 20 | | 20 | | 20 | | 20 |
| 4 | 13 | Soluble PnP 14, 2 μg | NT | NT | NT | NT | NT | NT | NT | NT |
| | 14 | | | NT | | NT | | NT | | NT |
| | 15 | | | NT | | NT | | NT | | NT |
| | 16 | | | NT | | NT | | NT | | NT |
| | 17 | | | NT | | NT | | NT | | NT |
| | 18 | | | NT | | NT | | NT | | NT |
| 5 | 19 | Prevnar ™ - 16 μg dose of PnP | 20 | 20 | 20 | 20 | 806 | 640 | 1810 | 2560 |
| | 20 | (2 μg per serotype) | | 20 | | 20 | | 1280 | | 5120 |
| | 21 | | | 20 | | 20 | | 5120 | | 10240 |
| | 22 | | | 20 | | 20 | | 1280 | | 2560 |
| | 23 | | | 20 | | 20 | | 40 | | 80 |
| | 24 | | | 20 | | 20 | | 1280 | | 1280 |
| 6 | 25 | 80 μg 80 × 360 nm PRINT ™ | 25 | 40 | 36 | 40 | 32 | 40 | 40 | 40 |
| | 26 | 5% DMAEMA/PLGA + 2 μg | | 20 | | 40 | | 20 | | 40 |
| | 27 | PnP 4 | | 20 | | 40 | | 20 | | 40 |
| | 28 | | | 20 | | 40 | | 40 | | 40 |
| | 29 | | | 40 | | 40 | | 40 | | 40 |
| | 30 | | | 20 | | 20 | | 40 | | 40 |
| 7 | 31 | 120 μg 80 × 360 nm PRINT ™ | 25 | 40 | 25 | 40 | 28 | 40 | 28 | 40 |
| | 32 | 5% DMAEMA/PLGA + 2 μg | | 20 | | 20 | | 20 | | 20 |
| | 33 | PnP 4 | | 20 | | 20 | | 40 | | 40 |
| | 34 | | | 20 | | 20 | | 20 | | 20 |
| | 35 | | | 40 | | 20 | | 20 | | 40 |
| | 36 | | | 20 | | 40 | | 40 | | 20 |
| 8 | 37 | 80 μg 80 × 360 nm PRINT ™ | 40 | 40 | 32 | 40 | 40 | 40 | 36 | 40 |
| | 38 | 5% DMAEMA/PLGA + 2 μg | | 40 | | 40 | | 40 | | 40 |
| | 39 | PnP 4 + 2 μg CRM197 | | 40 | | 40 | | 40 | | 40 |
| | 40 | | | 40 | | 40 | | 40 | | 40 |
| | 41 | | | 40 | | 20 | | 40 | | 20 |
| | 42 | | | 40 | | 20 | | 40 | | 40 |
| 9 | 43 | 120 μg 80 × 360 nm PRINT ™ | 22 | 20 | 40 | 20 | 28 | 20 | 36 | 20 |
| | 44 | 5% DMAEMA/PLGA + 2 μg | | 20 | | 40 | | 40 | | 40 |
| | 45 | PnP 4 + 2 μg CRM197 | | 20 | | 20 | | 20 | | 20 |
| | 46 | | | 20 | | 40 | | 20 | | 40 |
| | 47 | | | 40 | | 40 | | 40 | | 40 |
| | 48 | | | 20 | | 160 | | 40 | | 80 |
| 12 | 61 | 60 μg 200 × 200 nm albumin- | NT | NT | NT | NT | NT | NT | NT | NT |
| | 62 | based particles with 1.5 μg* | | NT | | NT | | NT | | NT |
| | 63 | PnP 14 | | NT | | NT | | NT | | NT |
| | 64 | | | NT | | NT | | NT | | NT |
| | 65 | | | NT | | NT | | NT | | NT |
| | 66 | | | NT | | NT | | NT | | NT |
| 13 | 67 | 60 μg 200 × 200 nm albumin- | 20 | 20 | 20 | 20 | 20 | 20 | 20 | 20 |
| | 68 | based particles with 1.5 μg* | | 20 | | 20 | | 20 | | 20 |
| | 69 | CRM 197 and 1.5 μg* PnP 14 | | 20 | | 20 | | 20 | | 20 |
| | 70 | | | 20 | | 20 | | 20 | | 20 |
| | 71 | | | 20 | | 20 | | 20 | | 20 |
| | 72 | | | NT | | NT | | NT | | NT |
| 14 | 73 | 120 μg 200 × 200 nm | 20 | 20 | 20 | 20 | 32 | 40 | 45 | 160 |
| | 74 | albumin-based particles with 3 μg* | | 20 | | 20 | | 20 | | 20 |
| | 75 | CRM 197 and 3 μg* PnP 4 | | 20 | | 20 | | 20 | | 20 |
| | 76 | | | 20 | | 20 | | 20 | | 20 |
| | 77 | | | 20 | | 20 | | 160 | | 320 |
| | 78 | | | 20 | | 20 | | 20 | | 20 |

| | | | PnP 14 | | | | | | | |
|---|---|---|---|---|---|---|---|---|---|---|
| | | | Pre-bleeds | | 4 week PP | | 1 week PB | | 2 weeks PB | |
| Group | Animal | Vaccine | GMT | Titers | GMT | Titers | GMT | Titers | GMT | Titers |
| 1 | 1 | Vehicle Control | 20 | 20 | 20 | 20 | 20 | 20 | 20 | 20 |
| | 2 | | | 20 | | 20 | | 20 | | 20 |
| | 3 | | | 20 | | 20 | | 20 | | 20 |
| 2 | 4 | 80 μg 80 × 360 nm PRINT | 20 | 20 | 20 | 20 | 20 | 20 | 20 | 20 |
| | 5 | 5% DC-Chol/PLGA, no | | 20 | | 20 | | 20 | | 20 |
| | 6 | protein | | 20 | | 20 | | 20 | | 20 |
| 3 | 7 | Soluble PnP 4, 2 μg | NT | NT | NT | NT | NT | NT | NT | NT |
| | 8 | | | NT | | NT | | NT | | NT |
| | 9 | | | NT | | NT | | NT | | NT |
| | 10 | | | NT | | NT | | NT | | NT |
| | 11 | | | NT | | NT | | NT | | NT |
| | 12 | | | NT | | NT | | NT | | NT |
| 4 | 13 | Soluble PnP 14, 2 μg | 20 | 20 | 20 | 20 | 20 | 20 | 20 | 20 |
| | 14 | | | 20 | | 20 | | 20 | | 20 |
| | 15 | | | 20 | | 20 | | 20 | | 20 |
| | 16 | | | 20 | | 20 | | 20 | | 20 |
| | 17 | | | 20 | | 20 | | 20 | | 20 |
| | 18 | | | 20 | | 20 | | 20 | | 20 |
| 5 | 19 | Prevnar ™ - 16 μg dose of PnP | 20 | 20 | 20 | 20 | 63 | 20 | 202 | 2560 |
| | 20 | (2 μg per serotype) | | 20 | | 20 | | 20 | | 20 |
| | 21 | | | 20 | | 20 | | 10240 | | 20480 |
| | 22 | | | 20 | | 20 | | 20 | | 20 |
| | 23 | | | 20 | | 20 | | 20 | | 20 |
| | 24 | | | 20 | | 20 | | 40 | | 160 |
| 10 | 49 | 120 μg 80 × 360 nm PRINT ™ | 20 | 20 | 20 | 20 | 20 | 20 | 20 | 20 |
| | 50 | 5% DMAEMA/PLGA + 2 μg | | 20 | | 20 | | 20 | | 20 |
| | 51 | PnP 14 | | 20 | | 20 | | 20 | | 20 |
| | 52 | | | 20 | | 20 | | 20 | | 20 |
| | 53 | | | 20 | | 20 | | 20 | | 20 |
| | 54 | | | 20 | | 20 | | 20 | | 20 |
| 11 | 55 | 120 μg 80 × 360 nm PRINT ™ | 20 | 20 | 20 | 20 | 20 | 20 | 20 | 20 |
| | 56 | 5% DMAEMA/PLGA + 2 μg | | 20 | | 20 | | 20 | | 20 |
| | 57 | PnP 14 + 2 μg CRM197 | | 20 | | 20 | | 20 | | 20 |
| | 58 | | | 20 | | 20 | | 20 | | 20 |
| | 59 | | | 20 | | 20 | | 20 | | 20 |
| | 60 | | | 20 | | 20 | | 20 | | 20 |
| 12 | 61 | 60 μg 200 × 200 nm albumin- | 20 | 20 | 285 | 160 | 905 | 640 | 2874 | 2560 |
| | 62 | based particles with 1.5 μg* | | 20 | | 160 | | 640 | | 2560 |
| | 63 | PnP 14 | | 20 | | 320 | | 1280 | | 5120 |
| | 64 | | | 20 | | 640 | | 1280 | | 5120 |
| | 65 | | | 20 | | 320 | | 640 | | 1280 |
| | 66 | | | 20 | | 320 | | 1280 | | 2560 |
| 13 | 67 | 60 μg 200 × 200 nm albumin- | 20 | 20 | 113 | 320 | 226 | 640 | 1810 | 2560 |
| | 68 | based particles with 1.5 μg* | | 20 | | 80 | | 40 | | 320 |
| | 69 | CRM 197 and 1.5 μg* PnP 14 | | 20 | | 160 | | 1280 | | 10240 |
| | 70 | | | 20 | | 20 | | 20 | | 640 |
| | 71 | | | 20 | | 80 | | 640 | | 5120 |
| | 72 | | | 20 | | 320 | | 320 | | 1280 |
| 14 | 73 | 120 μg 200 × 200 nm | 20 | 20 | 20 | 20 | 20 | 20 | 20 | 20 |
| | 74 | albumin-based particles with 3 μg* | | 20 | | 20 | | 20 | | 20 |
| | 75 | CRM 197 and 3 μg* PnP 4 | | 20 | | 20 | | 20 | | 20 |
| | 76 | | | 20 | | 20 | | 20 | | 20 |
| | 77 | | | 20 | | 20 | | 20 | | 20 |
| | 78 | | | 20 | | 20 | | 20 | | 20 |

Follow-up (Liq. 27 ELISA (IgG))

| | | | | PnP4 | | | | | |
|---|---|---|---|---|---|---|---|---|---|
| | | | | 4 week PP | | 1 week PB | | 2 week PB | |
| | | | | (post prime) | | (post boost) | | (post boost) | |
| Group | Animal | Route | Vaccine | IgG GMT | IgG Titer | IgG GMT | IgG Titer | IgG GMT | IgG Titer |
| 1 | 1 | SC | Blank OVA particles | 20.0 | 20 | 20 | 20 | 20 | 20 |
| | 2 | | | | 20 | | 20 | | 20 |
| | 3 | | | | 20 | | 20 | | 20 |
| 5 | 22 | SC | Prevnar ™ 16 μg dose of | 67.3 | 20 | 2560 | 40 | 13280 | 2560 |
| | 23 | | PnP, 2 μg per serotype | | 20 | | 2560 | | 20480 |
| | 24 | | | | 20 | | 5120 | | 5120 |
| | 25 | | | | 640 | | 5120 | | 20480 |
| | 26 | | | | 20 | | 5120 | | 20480 |
| | 27 | | | | 40 | | 5120 | | 20480 |
| | 44 | | | | 160 | | 5120 | | 20480 |
| | 45 | | | | 640 | | 5120 | | 20480 |
| 6 | 28 | SC | 1st inj: Prevnar ™ 16 μg | 30.8 | 20 | 160 | 320 | 67 | 160 |
| | 29 | | dose of PnP, 2 μg per | | 20 | | 160 | | 40 |
| | 30 | | serotype, 2nd inj: 2 μg PnP | | 20 | | 20 | | 20 |
| | 31 | | 4, 2 μg PnP 14 | | 40 | | 640 | | 160 |
| | 32 | | | | 20 | | 160 | | 80 |
| | 33 | | | | 20 | | 40 | | 20 |
| | 50 | | | | 320 | | 2560 | | 640 |
| | 51 | | | | 20 | | 40 | | 20 |
| 11 | 58 | SC | 120 μg 200 nm PRINT ™ | 20.0 | 20 | 20 | 20 | 20 | 20 |
| | 59 | | OVA + 2 μg PnP 4 | | 20 | | 20 | | 20 |
| | 60 | | (solution cross-linked) | | 20 | | 20 | | 20 |
| | 61 | | | | 20 | | 20 | | 20 |
| 13 | 70 | SC | PneumoVax | 20.0 | 20 | 20 | 20 | 20 | 20 |
| | 71 | | | | 20 | | 20 | | 20 |
| | 72 | | | | 20 | | 20 | | 20 |
| | 73 | | | | 20 | | 20 | | 20 |
| | 74 | | | | 20 | | 20 | | 20 |
| | 75 | | | | 20 | | 20 | | 20 |
| | 62 | | | | 20 | | 20 | | 20 |
| | 63 | | | | 20 | | 20 | | 20 |
| | | | | PnP14 | | | | | |
| | | | | 4 week PP | | 1 week PB | | 2 week PB | |
| | | | | (post prime) | | (post boost) | | (post boost) | |
| Group | Animal | Route | Vaccine | IgG GMT | IgG Titer | IgG GMT | IgG Titer | IgG GMT | IgG Titer |
| 1 | 1 | SC | Blank OVA particles | 20.0 | 20 | 20 | 20 | 20 | 20 |
| | 2 | | | | 20 | | 20 | | 20 |
| | 3 | | | | 20 | | 20 | | 20 |
| 2 | 4 | SC | Soluble PnP 14, 2 μg | 20.0 | 20 | 20 | 20 | 20 | 20 |
| | 5 | | | | 20 | | 20 | | 20 |
| | 6 | | | | 20 | | 20 | | 20 |
| | 7 | | | | 20 | | 20 | | 20 |
| | 8 | | | | 20 | | 20 | | 20 |
| | 9 | | | | 20 | | 20 | | 20 |
| 3 | 10 | SC | 120 μg soluble OVA, | 22.4 | 20 | 20 | 20 | 20 | 20 |
| | 11 | | Soluble PnP 14, 2 μg | | 20 | | 20 | | 20 |
| | 12 | | | | 20 | | 20 | | 20 |
| | 13 | | | | 20 | | 20 | | 20 |
| | 14 | | | | 20 | | 20 | | 20 |
| | 15 | | | | 40 | | 20 | | 20 |
| 4 | 16 | SC | 120 μg soluble OVA (EU- | 20.0 | 20 | 20 | 20 | 20 | 20 |
| | 17 | | free), Soluble PnP 14, 2 μg | | 20 | | 20 | | 20 |
| | 18 | | | | 20 | | 20 | | 20 |
| | 19 | | | | 20 | | 20 | | 20 |
| | 20 | | | | 20 | | 20 | | 20 |
| | 21 | | | | 20 | | 20 | | 20 |
| 5 | 22 | SC | Prevnar ™ 16 μg dose of | 25.9 | 20 | 160 | 40 | 320 | 160 |
| | 23 | | PnP, 2 μg per serotype | | 160 | | 640 | | 2560 |
| | 24 | | | | 20 | | 80 | | 160 |
| | 25 | | | | 20 | | 640 | | 640 |
| | 26 | | | | 20 | | 40 | | 80 |
| | 27 | | | | 20 | | 1280 | | 2560 |
| | 44 | | | | 20 | | 80 | | 160 |
| | 45 | | | | 20 | | 80 | | 80 |
| 6 | 28 | SC | 1st inj: Prevnar ™ 16 μg | 28.3 | 20 | 44 | 40 | 40 | 20 |
| | 29 | | dose of PnP, 2 μg per | | 20 | | 20 | | 20 |
| | 30 | | serotype, 2nd inj: 2 μg | | 20 | | 20 | | 20 |
| | 31 | | PnP 4, 2 μg PnP 14 | | 20 | | 20 | | 20 |
| | 32 | | | | 20 | | 20 | | 20 |
| | 33 | | | | 20 | | 20 | | 20 |
| | 50 | | | | 320 | | 5120 | | 5120 |
| | 51 | | | | 20 | | 20 | | 20 |
| 7 | 34 | SC | 120 μg 200 nm | 25.2 | 40 | 50 | 160 | 226 | 1280 |
| | 35 | | PRINT ™ OVA + 2 μg PnP | | 40 | | 80 | | 640 |
| | 36 | | 14 (array cross-link) | | 20 | | 40 | | 80 |
| | 37 | | | | 20 | | 20 | | 80 |
| | 38 | | | | 20 | | 80 | | 320 |
| | 39 | | | | 20 | | 20 | | 80 |
| 8 | 40 | SC | 120 μg 200 nm PRINT ™ | 23.8 | 40 | 57 | 160 | 269 | 320 |
| | 41 | | OVA (EU-free) + 2 μg PnP | | 20 | | 20 | | 320 |
| | 42 | | 14 (array cross-link) | | 20 | | 40 | | 40 |
| | 43 | | | | 20 | | 80 | | 1280 |
| 9 | 46 | SC | 1st inj: 120 μg 200 nm | 28.3 | 40 | 80 | 160 | 95 | 320 |
| | 47 | | PRINT ™ OVA (EU-free) + | | 40 | | 20 | | 320 |
| | 48 | | 2 μg PnP 14, 2nd inj: 2 μg | | 20 | | 40 | | 40 |
| | 49 | | PnP 14 | | 20 | | 320 | | 20 |
| 10 | 52 | SC | 120 μg 200 nm | 56.6 | 80 | 202 | 1280 | 905 | 5120 |
| | 53 | | PRINT ™ OVA (EU-free) + | | 20 | | 40 | | 160 |
| | 54 | | 2 μg PnP 14 (solution cross- | | 40 | | 80 | | 320 |
| | 55 | | linked) | | 160 | | 160 | | 1280 |
| | 56 | | | | 40 | | 320 | | 640 |
| | 57 | | | | 80 | | 320 | | 2560 |
| 12 | 64 | SC | 120 μg 200 nm PRINT ™ | 44.9 | 40 | 71 | 160 | 254 | 1280 |
| | 65 | | HSA + 2 μg PnP 14 | | 20 | | 40 | | 320 |
| | 66 | | (solution cross-linked) | | 20 | | 20 | | 320 |
| | 67 | | | | 20 | | 20 | | 40 |
| | 68 | | | | 160 | | 320 | | 320 |
| | 69 | | | | 160 | | 160 | | 160 |
| 13 | 70 | SC | PneumoVax | 20.0 | 20 | 20 | 20 | 20 | 20 |
| | 71 | | | | 20 | | 20 | | 20 |
| | 72 | | | | 20 | | 20 | | 20 |
| | 73 | | | | 20 | | 20 | | 20 |
| | 74 | | | | 20 | | 20 | | 20 |
| | 75 | | | | 20 | | 20 | | 20 |
| | 62 | | | | 20 | | 20 | | 20 |
| | 63 | | | | 20 | | 20 | | 20 |

LIQ27 ELISA (IgM) - Pre-bleeds & Cross Reactivity

| | | | | PnP14 | | | | | |
|---|---|---|---|---|---|---|---|---|---|
| | | | | | | 4 week PP | | 2 week PB | |
| | | | | Pre-Bleeds | | (post prime) | | (post boost) | |
| Group | Animal | Route | Vaccine | IgM GMT | IgMTiter | IgM GMT | IgMTiter | IgM GMT | IgM Titer |
| 10 | 52 | SC | 120 μg 200 nm PRINT ™ OVA [symbol] | 71 | 80 | 143 | 160 | 453 | 640 |
| | 53 | | [symbol] + 2 μg PnP 14 (solution | | 80 | | 80 | | 320 |
| | 54 | | cross-linked) | | 80 | | 320 | | 640 |
| | 55 | | | | [symbol] | | 160 | | 320 |
| | 56 | | | | 40 | | [symbol] | | 320 |
| | 57 | | | | [symbol] | | 160 | | 640 |
| 11 | 58 | SC | 120 μg 200 nm PRINT ™ OVA + | — | — | 67 | 40 | [symbol] | 160 |
| | 59 | | 2 μg PnP 4 (solution cross- | | — | | 40 | | 60 |
| | 60 | | [symbol] ) | | — | | 160 | | 320 |
| | 61 | | | | — | | [symbol] | | 320 |

[symbol]

indicates data missing or illegible when filed

1.-17. (canceled)

18. A method of making an immunogenic composition, comprising:
molding a substantially uniform plurality of particles comprising a biocompatible polymer;
introducing the molded plurality of particles to a composition comprising a protein and a polysaccharide; and
allowing the protein and polysaccharide to associate with a surface of the molded particles.

19. The method of claim 18, wherein the protein and polysaccharide are not cross-linked to each other or to the particle.

20. The method of claim 18 further comprising coating the particles after allowing the protein and polysaccharide to associate with the surface of the particle.

21. The method of claim 18, wherein the surface association comprises ionic interactions.

22. The method of claim 18, wherein the polysaccharide comprises less than 10 wt % of the particle.

23. The method of claim 18, wherein the protein comprises less than 10 wt % of the particle.

24. The method of claim 18 further comprising including a charged molecule with the biocompatible polymer of the particle composition.

25. A method of making multiple immunogenic compositions, comprising:
molding a substantially uniform plurality of particles comprising a biocompatible polymer;
separating the substantially uniform plurality of particles into a first and a second collection of particles;
introducing the first collection of molded particles to a composition comprising a protein and a first polysaccharide;
allowing the protein and first polysaccharide to associate with a surface of the first collection of particles;
introducing the second collection of molded particles to a composition comprising the protein and a second polysaccharide; and
allowing the protein and second polysaccharide to associate with a surface of the second collection of particles.

26. The method of claim 25, further comprising mixing the first and second collection of particle together in a vaccine.

27. The method of claim 25, wherein the surface association is not cross linked.

28. The method of claim 25, wherein the particle composition further comprises a charged molecule with the biocompatible polymer.

29. The method of claim 25, wherein the surface association comprises ionic interactions.

30. The method of claim 25, wherein the polysaccharide comprises less than 10 wt % of the particle.

31. The method of claim 25, wherein the protein comprises less than 10 wt % of the particle.

32. An immunogenic composition comprising:
a plurality of molded particles having substantially equivalent three-dimensional shape and substantially uniform composition;
wherein the substantially uniform composition comprises a biocompatible polymer; and
wherein a surface of the molded particle comprises a non cross linked protein and polysaccharide.

33. The immunogenic composition of claim 32, wherein the polysaccharide comprises less than 10 wt % of the particle.

34. The immunogenic composition of claim 32, wherein the wherein the protein is less than 10 wt % of the particle.

35. The immunogenic composition of claim 32, wherein the polymer is PLGA.

36. The immunogenic composition of claim 32, wherein the composition of the molded particle further comprising a charged molecule.

37. The immunogenic composition of claim 32, wherein each molded particle of the plurality of particles has the same broadest dimension and the broadest dimension is less than 10 micrometers.
